(19) United States
(12) Patent Application Publication
(10) Pub. No.: US 2005/0075542 A1
(43) Pub. Date: Apr. 7, 2005

(54) System and method for automatic monitoring of the health of a user

(76) Inventor: Rami Goldreich, HaAyin (IS)

Correspondence Address: Anthony Castorina;G E Ehrlich, Suite 207, 2001 Jefferson Davis Highway, Arlington, VA, 22202, US

(21) Appl. No.: 10/433,623

(22) PCT Filed: Dec. 21, 2001
(86) PCT No.: PCT/IL01/01187

Related U.S. Application Data
(60) Provisional application No. 60/258,042, filed on Dec. 27, 2000.

(51) Int. Cl. A61B 5/00

(52) U.S. Cl. 600/300

(57) ABSTRACT

A system and method for automatically monitoring at least one physiological function of the user, without active intervention by the user, in a non-invasive manner. Such monitoring may be used to detect a deterioration in the health of the user. Preferably, the system according to the present invention features at least one physiological sensor for measuring the physiological parameter of the user to obtain the measurement of a physiological function, a local processing unit for extracting medical information from the physiological measurement, and a main server for processing the medical information in order to evaluate the health of the user. Such an evaluation is preferably performed by comparing medical information which has been obtained from a plurality of physiological measurements. Optionally and more preferably, the user is alerted if the evaluation detects a deterioration in at least one physiological function.

### FIELD OF THE INVENTION

The present invention relates to a method and system for automatically monitoring the health of a user with at least one measuring device, and in particular, to such a system and method in which the measurements are performed automatically without the intervention of the user.

### DESCRIPTION OF THE BACKGROUND ART

Many different types of diseases are preventable or at least treatable if early detection of one or more symptoms or aspect of the disease is possible. Such early detection is currently performed by requiring the subject to receive regular examinations by a doctor, such as an annual examination for example. However, even annual examinations may not be sufficiently frequent in order to detect early signs of disease, yet requiring more frequent examinations could result in reduced compliance of the subject and increased cost.

One example of a disease for which more frequent monitoring could be useful is cardiac disease. Early detection of symptoms of cardiac disease, such as an increase in blood pressure, decrease in overall cardiac function, and/or development of a cardiac arrhythmia for example, could result in earlier and more effective treatment.

As is well known in the background art, monitoring a subject for one or more symptoms of heart disease is primarily based on the measurement of the vital signs of the subject, such as heart beat, the pattern of cardiac function such as arrhythmia, heart rate variability, ECG measurements, blood pressure, and optionally also body temperature and respiration parameters, at regular intervals. These measurement(s) are performed in order to ensure that the blood pressure level, heart beat rate and/or other aspects of cardiac function remain within the normal area.

However, in the present health care system it is not possible for financial and practical reasons for a person specialized in treating heart disease to personally monitor continuously the health of a subject. Therefore, as previously described, the subject must be examined periodically by medical personnel. However, periodic examinations may not be performed with sufficient frequency to detect a health problem and/or deterioration in the function of the body of the subject, until such deterioration has already become pronounced. A more effective type of examination would therefore allow the subject to perform at least some aspects of the examination outside of a medical environment, without direct assistance from medical personnel, for example at home.

In order to perform such an examination at home, the subject would need to obtain one or more measurements. Currently, the subject needs to use a medical instrument, such as a manual or an automatic blood pressure inflating cuff device. Blood pressure measurements are usually performed by the home (non-medical) subject once a day. Such medical instruments are difficult and awkward for the subject to operate, such that the subject compliance may be reduced. Furthermore, the measurements can currently only be performed manually, such that the active intervention of the user is required. Thus, such measurements are not typically performed on a regular basis by individuals who are not known to be suffering from reduced cardiac function.

On other hand, regular monitoring of one or more vital signs, for example on a daily or weekly schedule, without interfering with the normal habits of the subject and/or becoming a nuisance to the subject, is clearly helpful for monitoring the health condition of the subject and to alert the subject in case of deterioration in the health of the subject. From the health care system point of view, it is a method to filter the needed users from the rest of the healthy population, so they could receive medical treatment as soon as the symptoms are detected; saving hospitalization days by implementing preventive medication for those needed users.

### SUMMARY OF THE INVENTION

The background art does not teach or suggest a system or method for automatically monitoring the health of the user, without requiring active intervention by the user. Furthermore, the background art does not teach or suggest a mechanism for automatically monitoring at least one physiological function of the user. The background art also does not teach or suggest such a mechanism, which can be easily operated outside of the medical environment. Such a system or method would clearly be useful, as it would enable the health of the user to be monitored frequently, thereby enabling earlier detection of a deterioration in the health of the user, with the possibility of early treatment.

The present invention overcomes these deficiencies of the background art by providing a system and method for automatically monitoring at least one physiological function of the user, without active intervention by the user, in a non-invasive manner. Such monitoring may be used to detect a deterioration in the health of the user. Preferably, the system according to the present invention features at least one physiological sensor for measuring at least one physiological parameter of the user, a local processing unit for extracting medical information by measuring at least one physiological function of the human body according to information obtained from the measurements, and a main server for processing the medical information in order to evaluate the health of the user. Such an evaluation is preferably performed by comparing medical information, which has been obtained from a plurality of physiological measurements. Optionally and more preferably, the user is alerted if the evaluation detects a deterioration in at least one physiological function.

According to a preferred embodiment of the present invention, the physiological measurements and/or the obtained medical information are stored in a database. Optionally and more preferably, such stored data is provided to medical personnel who are treating the user, for example for more accurate diagnosis. Also optionally and more preferably, medical personnel receive an alert if a deterioration in one or more physiological functions is detected.

Examples of physiological functions and medical information which may optionally be monitored by the present invention include, but are not limited to: heart rate, arrhythmia, heart rate variability, ECG, blood pressure, body temperature and respiration rate. As used herein, the term “physiological parameter” refers to a signal which is received from a sensor and/or medical instrument, while the term “medical information” refers to the information which may be extracted or otherwise obtained by analyzing this signal and/or a combination of signals.

One or more physiological sensors for monitoring the user according to the present invention may optionally be concealed in a device, which is normally used by the user as part of daily life. Such a device is preferably operated by the user for at least one function which is not related to monitoring a physiological function of the user. Examples of such devices include, but are not limited to, a watch, bracelet, cellular telephone, regular telephone connected to the PSTN (public switched telephone network), furniture such as a chair or bed for example, keyboard, computer mouse, computer mouse pad, and so forth. Therefore the measurements are performed without the requirement for direct action or intervention by the user, and hence with little or no interference with the user's daily life.

According to a preferred embodiment of the present invention, the physiological sensor which performs the physiological measurement is preferably connected to a local data processing unit through a communication component. The communication component preferably features wireless transmission, although alternatively the connection may be wired, through a cable for example. The local processor is itself more preferably connected to a main server, optionally through a wireless communication link but alternatively through a wired communication link.

The main server optionally and preferably features a database for storing the medical information and/or physiological measurements obtained from the local processor and/or the physiological sensor. The main server more preferably also features a software module for monitoring the user's health by performing an algorithm to issue an alert whenever necessary. The algorithm operates on data stored in the database, preferably to create a user medical profile, which is optionally and more preferably based on the user's medical history, medical information from external systems and on an average readings of physiological parameters, most preferably collected over an extended period of time, or at least collected repeatedly.

According to an optional implementation of the present invention, the system further features a medical service center that can optionally and preferably initiate a medical examination in order to obtain “on-line” or “real time” measurements of physiological parameters regarding the user's current medical status and to obtain an on-line report about recent and/or historical measurements. The medical report can also optionally and preferably be initiated also by the user, on-line via the Internet or other network for example, or off-line by any other communication means. Periodical reports regarding the user's measurements results are optionally and preferably sent to the user and/or to the medical service center.

The expression “medical service center” refers in this connection to anyone who participates in the monitoring of the user and who needs to monitor the development of the user's health. Therefore this person does not necessarily have to be a medical doctor, but should be qualified to work in a medical service center.

According to a preferred embodiment of the present invention, any significant deviation in measurements of a physiological parameter and/or medical information of the user from an expected standard causes an alert to be transmitted, optionally to the user, and alternatively or additionally to the medical service center and/or other medical personnel. The expected standard may optionally be relative to previous measurements of physiological parameters and/or previously obtained medical information. Alternatively or additionally, the expected standard may be absolute, such that the measurements are beyond the normal expected values, such as very high or very low blood pressure, arrhythmia, and so forth. The alert could optionally be sent to the medical service center in order to make a decision whether the user should contact a medical doctor for further medical examinations. Alerting the user could optionally be made by any kind of communication means (such as a voice message by telephone and/or sending a SMS or other text message to the cellular telephone, or by e-mail).

The invention also optionally and preferably relates to a portable measuring device with which the method according to the invention can be applied. The measuring device according to the invention is preferably characterized in that the measuring device features a measuring unit, an optional processing unit and a communications device that uses a wired or a wireless data transmission link. The measuring unit and/or the optional processing unit also preferably features some type of mechanism for supplying the results via the communications device to a system on a main server for data storage and processing, and optionally also for generating alerts, such that the data is more preferably also available to a medical service center.

The term “wired communications device” refers in this connection to any device which is suitable for wired communications and by means of which the user can transmit his measurement results to the data processing, storing and alerting system on a main server. Such a communications device may be for example any wired communication infrastructure, such as a PSTN, ISDN, Internet, LAN, cable modems and fiber-optic networks, etc.

The term “wireless communications device” refers in this connection to any device which is suitable for wireless communications and by means of which the user can transmit his measurement results to the data processing, storing and alerting system on a main server, regardless of where the user is at the moment. Such a communications device may be for example any radio transmitter, and/or mobile phone, Bluetooth device, wireless LAN, pager, etc.

The term “physiological sensor” refers in this connection to any sensor, optionally with a processing unit, which is suitable for measuring the physiological vital signs of the user or any standard medical equipment (such as automatic blood pressure device, ECG device and so forth, for example), that is capable of delivering output signal(s) and/or processed data via a data line or wireless link to the system on a main server and/or to a local data processing unit. Non-limiting, illustrative examples of such a sensor include a piezoceramic transducer, a piezoelectric transducer, a bio-impedance meter, a resistive strain gauge and a pressure sensor with fiber-optic components.

Among the advantages of the present invention are optionally and preferably the constant daily/weekly scheduled transmission of measurement results from the user to the server, the gathering of measurement results in the user's normal environment and the possibility for the server to monitor the recent development of the user's health without a visit to the doctor, in which case the user can visit the doctor/hospital only when required and not according to a predetermined schedule.

### BRIEF DESCRIPTION OF THE DRAWINGS

The foregoing and other objects, aspects and advantages will be better understood from the following detailed description of a preferred embodiment of the invention with reference to the drawings, wherein:

FIG. 1 is a schematic block diagram of an exemplary but preferred implementation of the system according to the present invention;

FIG. 2 shows a first exemplary implementation of the monitoring device according to the present invention; and

FIG. 3 shows a second exemplary implementation of the monitoring device according to the present invention.

### DESCRIPTION OF THE PREFERRED EMBODIMENTS

The present invention is of a system and method for automatically monitoring at least one physiological function of the user, without active intervention by the user, in a non-invasive manner. Such monitoring may be used to detect a deterioration in the health of the user. Preferably, the system according to the present invention features at least one physiological sensor for measuring the physiological parameter of the user to obtain the measurement of a physiological function, a local processing unit for extracting medical information from the physiological measurement, and a main server for processing the medical information in order to evaluate the health of the user. Such an evaluation is preferably performed by comparing medical information which has been obtained from a plurality of physiological measurements. Optionally and more preferably, the user is alerted if the evaluation detects a deterioration in at least one physiological ftnction.

Examples of physiological functions and medical information which may optionally be monitored by the present invention include, but are not limited to, heart beat, arrhythmia, heart rate variability, ECG, blood pressure, body temperature and respiration parameters.

One or more physiological sensors for monitoring the user according to the present invention may optionally be concealed in a device which is normally used by the user. Such a device is preferably operated by the user for at least one function which is not related to monitoring a physiological function of the user. Examples of such devices include, but are not limited to, a watch, bracelet, cellular telephone, regular telephone connected to the PSTN (public switched telephone network), furniture such as a chair or bed for example, keyboard, computer mouse, computer mouse pad, and so forth. Therefore the measurements are performed without a direct action or intervention by the user, and hence with little or no interference with the user's daily life.

According to a preferred embodiment of the present invention, the physiological sensor which performs the physiological measurement is preferably connected to a local data processing unit through a communication component. The communication component preferably features wireless transmission, although alternatively the connection may be wired, through a cable for example. The local processor is itself more preferably connected to a main server, optionally through a wireless connection but alternatively through a wired connection.

The main server optionally and preferably features a database for storing the medical information and/or physiological measurements obtained from the local processor. The main server more preferably also features a software module for monitoring the user's health by performing an algorithm to issue an alert whenever necessary. The algorithm operates on data stored in the database, preferably to create a user medical profile, which is optionally and more preferably based on the user's medical history, medical information from external systems and on an average readings of physiological parameters, most preferably collected over an extended period of time, or at least collected repeatedly.

The principles and operation of a device and method according to the present invention may be better understood with reference to the drawings and the accompanying description.

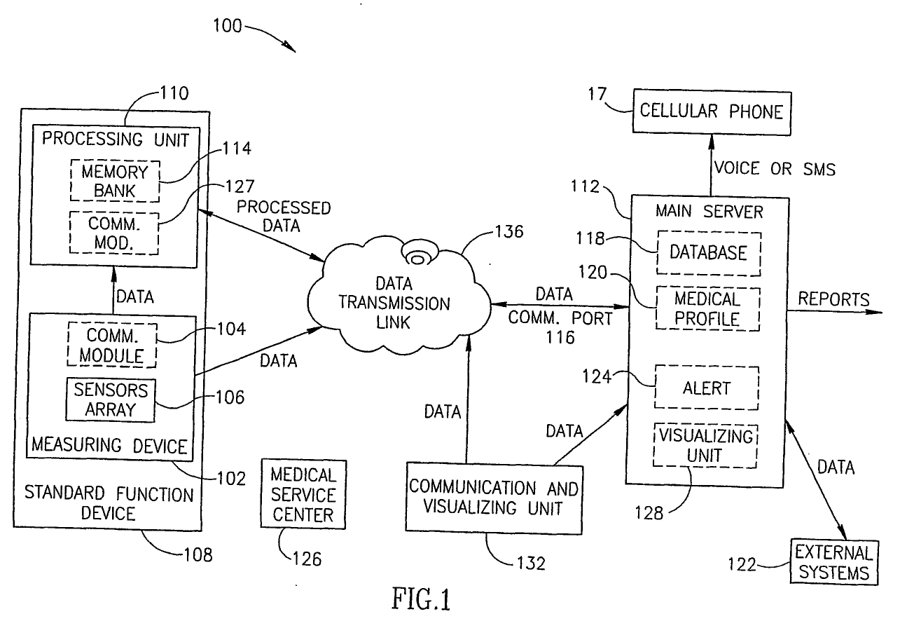

Turning now to the drawings, FIG. 1 is a block diagram of the preferred embodiment of the system according to the invention. A system 100 features a measuring device 102 for measuring at least one physiological parameter of the user. Measuring device 102 preferably features a communication module 104 and at least one physiological sensor 106, but more preferably features an array of physiological sensors as shown. Physiological sensor 106 senses at least one physiological parameter such as heart beat, arrhythmia, heart rate variability, ECG, blood pressure, body temperature and respiration parameters for example. Additionally or alternatively, physiological sensor 106 may also perform some other medically related measurement, such as measuring SpO2 (oxygen pressure in the blood) for example.

Measuring device 102 is preferably built into a device which is frequently used by the user in everyday tasks such as watch, bracelet, cellular phone, telephone, chair, keyboard, computer's mouse, computer's mouse pad, bed, etc. This device may be described as a standard function device 108. Therefore, during normal operation of standard function device 108 by the user, direct physical contact is maintained with the measuring device 102, preferably without the requirement for direct intervention or action by the user. One or more measurements may optionally be taken by measuring device 102 from the user automatically through such direct physical contact.

One optional but preferred example of measuring device 102 is a portable device which is preferably worn on the wrist of the user. For this example, standard function device 108 is preferably a wristwatch. According to preferred embodiments of the present invention, the wrist-mounted device (measuring device 102 with standard function device 108) features one or more sensors attached to a wristband or other fastening article. The sensor(s) are preferably connected to a microprocessor, optionally by a wire but alternatively through a wireless connection. The microprocessor may optionally also be located within the wristband, or otherwise attached to the wristband. The sensor(s) preferably support automatic collection of at least one physiological measurement; more preferably, the microprocessor is able to execute one or more instructions for extracting clinically useful information about the user from such measurement(s).

The microprocessor more preferably operates a software program to process and analyze the data which is collected, in order to compute medical information. The extracted medical information, optionally also with the raw data, is then preferably transferred to the previously described communication module. This module then preferably relays such information to a main server, which more preferably is able to provide such information to medical personnel, for example as part of a medical service center. Therefore, continuous monitoring of the physiological parameters of the user may optionally and more preferably be made, enabling better medical care for the user.

A general, non-limiting example of suitable formulae for measuring the heart rate and/or other heart-related physiological parameters of a subject who is wearing the device according to the present invention may be found in the article “Cuff-less Continuous Monitoring of Beat-To-Beat Blood Pressure Using Sensor Fusion”, by Boo-Ho Yang, Yi Zhang and H. Harry Asada—IEEE (also available through http://web.mit.edu/zyi/www/pdf/IEEETrans2000.pdf as of Dec. 9, 2001), hereby incorporated by reference as if fully set forth herein, where systolic and diastolic blood pressure are calculated using the pulse pressure shape per heartbeat. The disclosure does not describe a device which has the functionality according to the present invention, but the disclosed method is generally useful for determining blood pressure from an external measurement of pressure from the pulse through the skin of the subject.

After the measurement has been performed, communication module 104 preferably transmits the measurement result to a local data processing unit 110. Communication module 104 may optionally be a wired or wireless communication such as serial communication port (using serial protocols such as RS232, IRda or USB) or “Bluetooth” communication controller. Communication module 104 then preferably transmits the measurement result supplied by physiological sensor 106, for example in the form of a data packets, to local processing unit 110. A similar communication module 127 also performs communication at local processing unit 110, and is of a corresponding, compatible type to the type of communication module 104. Local data processing unit 110 may also optionally be incorporated within standard function device 108 as shown, or alternatively may be incorporated in a separate device (not shown). Measuring device 102 and local data processing unit 110 can therefore optionally and preferably be combined in a single enclosure, whether as part of standard function device 108 or otherwise, thereby creating a stand-alone medical device, which includes both measuring and processing functions.

The transmitted data is optionally and preferably sent, additionally or alternatively, directly to a main server 112. According to an optional embodiment, one or both of communication module 104 (if the measured data of physiological sensor 106 is transmitted directly to main server 112, as described in greater detail below) or communication module 127 may optionally be implemented as a mobile unit (such as a cellular telephone) which transmits the measurement result supplied by physiological sensor 106, optionally using the telephone as a cellular modem (i.e. sending data in the form of cellular data packets) or alternatively in form of a Short Message Service (SMS) message, or any other suitable format.

For the preferred embodiment in which local data processing unit 110 receives the data, local data processing unit 110 preferably first decodes the message to extract the sensor data. Local data processing unit 110 then preferably executes an algorithm to extract medical information, such as heart beat rate, arrhythmia, heart rate variability and/or divergence of the pattern of heartbeats over a period of time, calculating the blood pressure from a blood pulse pressure sensor and/or calculating the respiration rate for example, or any combination thereof. As previously described, preferably an algorithm is taken from the article “Cuff-less Continuous Monitoring of Beat-To-Beat Blood Pressure Using Sensor Fusion”, by Boo-Ho Yang, Yi Zhang and H. Harry Asada—IEEE (also available through http://web.mit.edu/zyi/www/pdf/IEEETrans2000pdf as of Dec. 9, 2001), previously incorporated by reference.

Local data processing unit 110 optionally and preferably stores the sensor data and the calculated results in a memory 114. More preferably, local data processing unit 110 stores the data and calculated results at least until this information is to be transmitted to main server 112 through a communication module 127.

Once received by main server 112, the data is preferably first added to a database 118. Once a plurality of such measurements of physiological parameters and/or medical information has been collected, main server 112 preferably executes an algorithm to create a medical profile 120 for the user. Medical profile 120 optionally and more preferably also incorporates information gathered from external medical server and databases. Examples of such information include but are not limited to the medical history of the user and medical information from an external system 122. External system 122 may optionally be a different medical instrument or database, for example hospital records stored in a database. Additionally or alternatively, medical profile 120 preferably includes information obtained by combining average readings of physiological parameters, and more preferably includes their divergence, collected over an extended period of time by measuring device 102.

The operation of the algorithm by main server 112 preferably enables any alteration, change or deterioration in the physiological function of the user to be determined, by comparing recent measurements of one or more physiological parameters with information taken from medical profile 120. Optional but preferred examples of comparisons which could be performed include but are not limited to detecting any increase in average readings of systolic blood pressure over time in comparison to average recent readings of systolic blood pressure, and/or any alteration in average heart rate, especially outside the normal range. Optionally and more preferably, such a determination of an alteration, change or deterioration in the condition of the user causes main server 112 to activate an alert module 124. Alert module 124 preferably causes an alert message to be sent directly to the user and/or to a medical service center 126.

Preferably, any readings beyond the normal expected values (such as very high or very low blood pressure), which may represent a dangerous medical situation for the user also activate alert module 124.

The alert message could optionally be sent to medical service center 126 to review the measurements of the physiological parameters in order to determine whether the user and/or the personnel at medical service center 126 should contact a medical doctor and/or emergency services.

The user may optionally be alerted through any suitable communication mechanism, such as voice communication and/or message by telephone, an SMS message to a cellular telephone 130, an alert message to local processing unit 110 (in cases where it has a display or any kind of audible alert) or an e-mail message. Such an alert message preferably includes a request for the user to be examined by a medical doctor and/or another type of request for intervention by trained medical personnel.

Optionally and preferably, the medical doctor is also able to retrieve the medical data stored in main server 112, more preferably by using a communication and visualizing unit 132 (such as a personal computer with a screen and a dial-up modem for contacting main server 112 and for retrieving information therefrom), in order to obtain further information for producing a more accurate diagnosis. Therefore the doctor (or other medical personnel) who is treating the user preferably always has access to the user's measurement results, regardless of the current location of the doctor and/or the user.

Personnel at medical service center 126 may optionally and preferably check the measurements using a visualization module 128 (such as a PC (personal computer) or a computer workstation with a screen to view the retrieved information as graphs and/or text, for example). Medical service center 126 can initiate a medical examination in order to obtain on-line physiological data regarding the physiological parameters of the user who is in physical contact with measuring device 102. Medical service center 126 can optionally receive such on-line data by first receiving an on-line message from the measuring device 102 that the user is currently in direct physical contact with measuring device 102. Next, then the medical service center 126 can optionally and preferably command measuring device 102 to take a measurement, and more preferably can receive the results immediately after finishing the examinations.

Medical service center 126 preferably defines and updates the services provided through measuring device 102 according to the medical results of the user, for example if the assessed medical information shows that the user is required to receive an alert, measuring device 102 is preferably commanded to take more measurements, for example at a greater frequency to be able to monitor the user more accurately. Periodical reports regarding the physiological measurements of the user are preferably sent to the user and/or to medical service center 126.

As previously described, medical service center 126 may optionally remotely initiate a medical examination in order to receive on-line physiological data regarding the user. Alternatively or additionally, such a check may optionally be manually initiated also by the user (for example by pressing on a start button while in direct contact with measuring device 102).

Periodical reports regarding these measurements results are optionally and preferably sent to the user and/or to medical service center 126. Also additionally or alternatively, reports may be received on-line, for example through the Internet, or “off-line” through any suitable communication mechanism.

Main server database 118 preferably contains, for each user, the results of the measurements, performed with measuring device 102 of the user and/or alternatively performed at the hospital (or other medical environment). The results are preferably stored in database 118, and are more preferably stored for an extended period of time, such as several years for example. Furthermore, the medical history of the user, as collected from external system 122, preferably is also available in database 118. Therefore, the doctor treating the user can optionally and preferably monitor the development of the user's health according to previous measurements, for example by using trend analysis even when the doctor and the user have not been in direct physical contact.

If required, the user may also transmit additional or alternative information, other than (or in addition to) the measurement result and the time of measurement from local processing unit 110. Thus, for example if local processing unit 110 is a computer, the user can chat (using a keyboard or a Voice Over IP method for example) or perform a video conference (using a digital camera with computer connection for example) with the medical doctor or medical service center 126, for example to supply additional data. Such additional data could optionally concern, for example, diet, dosage of medication, exercise or the like, any unusual or painful symptoms, and general feelings and/or symptoms. This information can also be forwarded through a data transmission link 136 to database 118 on main server 112.

Visualization module 128 preferably also provides other reports concerning individual users, such as periodical reports and/or special medical reports.

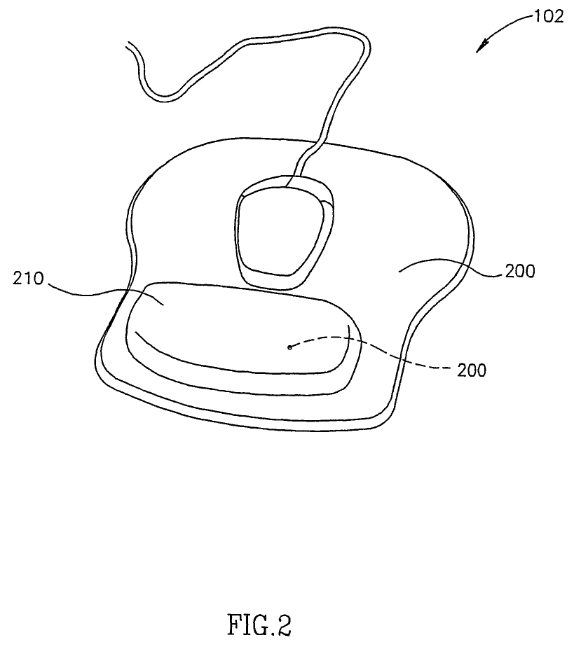

FIG. 2 illustrates an example of the preferred embodiment of the measuring device according to the invention. Measuring device 102 features a mouse pad 200 with the sensor or sensors (not shown) placed inside a cushion 210 of mouse pad 200. Communication module 104 (not shown) is concealed under cushion 210 with a battery case and a battery (also not shown) for providing the necessary power to measuring device 102.

Measuring device 102 is designed in such a way that it fits in the cushion space of mouse pad 200 when the normal filling of cushion 210 has been removed therefrom. In addition to the sensor(s), measuring device 102 therefore also comprises a battery that supplies an operating voltage to measuring device 102.

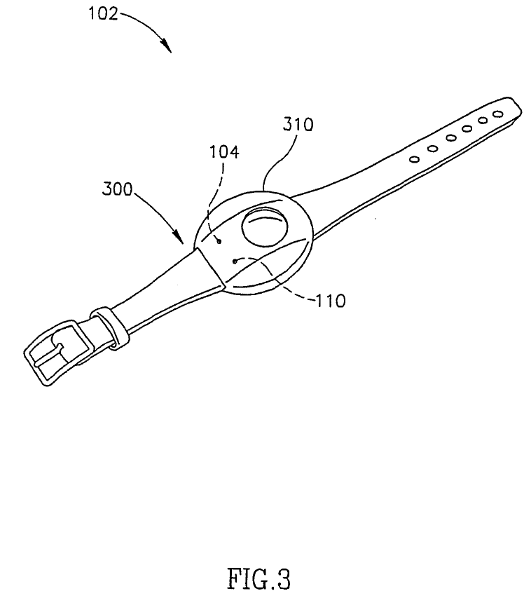

FIG. 3 illustrates an example of another preferred embodiment of the measuring device according to the invention. Measuring device 102 is now optionally implemented inside a panic-button bracelet 300 with the sensor or sensors (not shown) placed inside a case 310 of bracelet 300. Communication module 104 (not shown) and local processing unit 110 (also not shown) are concealed together in case 310 with a battery (also not shown) for providing the necessary power to measuring device 102 and local processing unit 110.

Measuring device 102 and local processing unit 110 (also not shown) are preferably designed in such a way that the combination has the approximate size of a wristwatch, wristband or a panic-button bracelet. The combination can optionally and preferably be used during normal use, for emergency tele-assistance, and/or preventive telemedicine, in order to detect deterioration of the user's health. In this example local processing unit 110 (also not shown) and communication module 104 (not shown) can optionally be installed, in addition to, or in place of, installation at bracelet 300, also in a cellular phone, by using any wireless communication, such as infrared, radio or a device enabled according to the Bluetooth communication protocol, between bracelet 300 and the cellular phone (not shown).

It will be appreciated that the above descriptions are intended only to serve as examples, and that many other embodiments are possible within the spirit and the scope of the present invention.

1-36. Cancel

37. A device for automatically measuring a physiological parameter of a user, and analyzing an associated physiological measurement of said user, in order to determine medical information, the device comprising:
(a) a sensor for automatically measuring said physiological parameter to form said physiological measurement upon intermittent physical contact of said sensor by the user, wherein said physiological measurement is processed to obtain user medical information about the user;
(b) a communication unit for transmitting said physiological measurement; and
(c) a standard function device for containing said sensor, wherein the user is in intermittent physical contact with said standard function device for at least one function other than measuring the physiological parameter of the user;
wherein said physiological measurement is processed to obtain user medical information about the user.

38. The device of claim 37, wherein the user's normal activities are not interrupted.

39. The device of claim 37, wherein active intervention is not required by the user.

40. The device of claim 37, wherein said measuring is noninvasive.

41. The device of claim 37, wherein said sensor comprises a piezoceramic transducer.

42. The device of claim 37, wherein said sensor comprises a piezoelectric transducer.

43. The device of claim 37, wherein said sensor comprises a bio-impedance meter.

44. The device of claim 37, wherein said sensor comprises a resistive strain gauge.

45. The device of claim 37, wherein said sensor comprises an electrical activity of the heart sensor.

46. The device of claim 37, wherein said sensor comprises a pressure sensor for measuring blood pressure.

47. The device of claim 37, wherein said sensor comprises a SpO2 sensor.

48. The device of claim 37, wherein said sensor comprises a sensor for measuring body temperature.

49. The device of claim 37, wherein said sensor comprises a pressure sensor with fiber-optic components.

50. The device of claim 37, wherein said standard function device is a computer mouse pad or a computer mouse.

51. The device of claim 37, wherein said standard function device is a watch.

52. The device of claim 37, wherein said standard function device is a bracelet.

53. The device of claim 37, wherein said standard function device is a cellular telephone.

54. The device of claim 37, wherein said standard function device is a regular telephone.

55. The device of claim 37, wherein said standard function device is a piece of furniture.

56. The device of claim 37, wherein said standard function device is a keyboard.

57. The device of claim 37, further comprising a local processor for analyzing said physiological measurement, wherein analyzing said physiological measurement by said local processor includes extracting medical information from said physiological measurement.

58. The device of claim 57, wherein analyzing said physiological measurement includes calculating systolic and diastolic blood pressure using pulse pressure shape.

59. The device of claim 37, wherein said physiological measurement includes at least one of heart rate and heart rate variability.

60. The device of claim 37, wherein said physiological measurement includes at least one of breathing rate and SPO2.

61. The device of claim 37, wherein said physiological measurement includes at least one of arrhythmia and overall cardiac rhythm.

62. The device of claim 37, wherein said physiological measurement includes at least one of body movements, an ECG measurement and blood pressure.

63. The device of claim 37, wherein said body movements include presence of abnormal body movements.

64. The device of claim 37, wherein said physiological measurement includes body temperature.

65. The device of claim 37, wherein said communication unit communicates according to a wired communication protocol.

66. The device of claim 37, wherein said communication unit communicates according to a wireless communication protocol.

67. The device of claim 66, wherein said wireless communication protocol is selected from the group consisting of an infrared communication protocol, a Bluetooth communication protocol, a wireless LAN and a radio communication protocol.

68. The device of claim 37, further comprising a manual activator for being activated by the user, such that measurement by said sensor is initiated upon manual activation.

69. The device of claim 37, wherein said intermittent physical contact is not continuous.

70. The device of claim 37, wherein said measurement is sampled and recorded as a function of time.

71. The device of claim 37, wherein the value of said measurement is compared against a normal range of values for said measurement.

72. The device of claim 37, wherein a comparison is made between the current value for said measurement and user's previous values for said measurement.

73. The device of claim 72, wherein an analysis of said comparison will register a deterioration in the user's health in the event said deterioration occurs.

74. The device of claim 37, further comprising a display for displaying information to the user.

75. The device of claim 74, wherein said display displays an alert to the user.

76. The device of claim 74, wherein said information comprises a result of at least one measurement.

77. A device for automatically measuring a physiological parameter of a user, and analyzing an associated physiological measurement of said user, in order to determine medical information, the device being operated in conjunction with a server, comprising:
(a) a sensor for automatically measuring said physiological parameter to form said physiological measurement upon intermittent physical contact of said sensor by the user; and
(b) a communication unit for transmitting said physiological measurement from said sensor to the server, wherein said physiological measurement is processed to obtain user medical information in order to evaluate the health of the user by the server, wherein the server analyzes said physiological measurement;
(c) a standard function device for containing said sensor, wherein the user is in intermittent physical contact with said standard function device for at least one function other than measuring the physiological parameter of the user.

78. The device of claim 77, wherein said sensor comprises a piezoceramic transducer.

79. The device of claim 77, wherein said sensor comprises a piezoelectric transducer.

80. The device of claim 77, wherein said sensor comprises a bio-impedance meter.

81. The device of claim 77, wherein said sensor comprises a resistive strain gauge.

82. The device of claim 77, wherein said sensor comprises an electrical activity of the heart sensor.

83. The device of claim 77, wherein said sensor comprises a pressure sensor for measuring blood pressure.

84. The device of claim 77, wherein said sensor comprises a SpO2 sensor.

85. The device of claim 77, wherein said sensor comprises a sensor for measuring body temperature.

86. The device of claim 77, wherein said sensor comprises a pressure sensor with fiber-optic components

87. A system for measuring and analyzing a physiological parameter of a user, comprising:
(a) a device for automatically measuring a physiological parameter of a user, and analyzing an associated physiological measurement of said user in order to determine medical information, the device comprising:
(i) a sensor for automatically measuring said physiological parameter to form said physiological measurement upon intermittent physical contact of said sensor by the user, wherein said physiological measurement is processed to obtain user medical information about the user;
(ii) a communication unit for transmitting said physiological measurement; and
(iii) a standard function device for containing said sensor, wherein the user is in intermittent physical contact with said standard function device for at least one function other than measuring the physiological parameter of the user; and
(b) at least one of a server, a medical service center or a communication and visualizing unit for receiving said physiological measurement from said device and for storing a plurality of long term physiological measurements.

88. The system of claim 87 further comprising a local processing unit, located at the user's premises, for receiving the physiological measurement from the measuring device and transmitting the physiological measurement to the server.

89. The system of claim 88 wherein the local processing unit analyzes the physiological measurement.

90. The system of claim 87, wherein the server analyzes the received physiological measurement.

91. The system of claim 89, wherein analyzing the received physiological measurement includes searching for deterioration in the physiological measurement of the user.

92. The system of claim 87 further comprising a medical service center wherein the server communicates with the medical service center for retrieving and transferring the stored physiological measurement to the medical service center.

93. The system of claim 91, wherein upon detecting deterioration in the physiological measurement of the user, the system sends an alert to the user and/or to the medical service center.

94. The system of claim 87, wherein only said server is present.

95. The system of claim 87 wherein said server is connected via a connection to a local processor.

96. The system of claim 95 wherein said connection is through a wireless communication link.

97. The system of claim 95 wherein said connection is through a wired communication link.

98. The system of claim 95 wherein said server optionally and preferably features a database for storing the medical information and/or physiological measurements obtained from the local processor and/or from the sensor.

99. The system of claim 98 wherein the server further comprises a software module for monitoring the user's health by performing an algorithm to issue an alert whenever necessary.

100. The system of claim 99 wherein a transmission of physiological measurement results is regularly scheduled to the server from the sensor operatively linked to the user.

101. The system of claim 100 wherein the server monitors recent developments in the user's health without a visit to the doctor.

102. The system of claim 101 wherein the server provides said physiological measurement results to medical personnel at a medical service center.

103. The system of claim 102 wherein the server executes an algorithm to create a medical profile for the user.

104. The system of claim 103 wherein the medical profile incorporates information gathered from an external medical server and at least one external database.

105. The system of claim 104 wherein the operation of the algorithm by the server preferably enables any alteration, change or deterioration in the physiological function of the user to be determined, by comparing recent measurements of one or more physiological measurements with information taken from the medical profile.

106. The system of claim 105 wherein a determination of an alteration, change or deterioration in the condition of the user causes the server to activate an alert module, which causes an alert message to be sent directly to the user and/or to a medical service center.

107. The system of claim 106 wherein medical personnel at the medical service center is able to retrieve the physiological measurement results stored in the server, by using a communication and visualizing unit in order to obtain further information for producing a more accurate diagnosis.

108. The system of claim 107 wherein the visualizing unit is a personal computer with a screen and a dial-up modem for contacting the server and for retrieving the physiological measurement results.

109. A method for automatically measuring a physiological parameter of a user, comprising:
providing a standard function device, comprising a sensor for measuring the physiological parameter;
physically contacting said standard function device by the user; and
automatically measuring the physiological parameter to form a physiological measurement upon said physical contact of the user with said standard function device.

110. A method for automatically measuring a physiological parameter of a user, comprising:
providing a standard function device, comprising a sensor for measuring the physiological parameter;
physically contacting said standard function device by the user; and
automatically measuring the physiological parameter to form a physiological measurement upon said physical contact of the user with said standard function device; and
alerting the user if said physiological measurement deviates from an acceptable standard.

111. A method for measuring a physiological parameter of a user, comprising:
providing a standard function device, comprising a sensor for measuring the physiological parameter, wherein said sensor is capable of being activated manually or automatically;
physically contacting said standard function device by the user; and
measuring the physiological parameter to form a physiological measurement upon said physical contact of the user with said standard function device, wherein said measuring is capable of being performed automatically upon said physical contact of the user with said standard function device and is also capable of being performed upon manual activation of said device by the user.

112. A device for automatically measuring a physiological parameter of a user, comprising:
(a) a sensor for automatically measuring the physiological parameter to form a physiological measurement upon physical contact of said sensor by the user, wherein said sensor is selected from the group consisting of a piezoceramic transducer, a piezoelectric transducer, a bio-impedance meter, a resistive strain gauge, an electrical activity of the heart sensor, a pressure sensor for measuring blood pressure, a SpO2 sensor, a sensor for measuring body temperature and a pressure sensor with fiber-optic components; and
(b) a communication unit for transmitting said physiological measurement.
